(19) United States
(12) Patent Application Publication
(10) Pub. No.: US 2005/0203592 A1
(43) Pub. Date: Sep. 15, 2005

(54) Irradiation device and use thereof

(76) Inventor: Klaus Teichert, Pullach (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/986,715

(22) Filed: Nov. 12, 2004

Related U.S. Application Data
(60) Provisional application No. 60/520,417, filed on Nov. 14, 2003.

(51) Int. Cl. A61N 5/06

(52) U.S. Cl. 607/88; 607/89

(57) ABSTRACT

An irradiation device (100) includes at least one laser light source (10) with a housing (11) wherein a laser device (12) is arranged, and a base unit (20) which includes a device body (21) and a control device (22), wherein the operating state of the laser device (12) can be set depending on control signals of the control device (22), and wherein the control device (22) is arranged so as to be separable from the device body (21).

### FIELD OF THE INVENTION

The invention relates to an irradiation device, in particular an irradiation device for laser-based acupuncture, tissue stimulation, surgery or materials processing, as well as the use of such an irradiation device. The invention also relates to a method for exposing an object to radiation.

### BACKGROUND OF THE INVENTION

There are a host of applications in biology and medicine which call for the exposure of biological tissue to radiation with ultraviolet, visible or infrared light. For example, methods for exposing tissue to laser light have been developed which are known by the name of “Low Level Laser Therapy” (LLLT). LLLT methods are in particular based on stimulating materials-related processes in cells or tissue by the admission of heat. Laser-based acupuncture is a further example, where at desired acupuncture points stimulation by laser irradiation takes place. Finally, laser-based surgery is known which features the cutting of tissue with a focused laser light analogous to the effect of a surgical scalpel.

From U.S. Pat. No. 5,464,436 an LLLT method is known in which an area to be treated is multiply exposed to laser-light radiation in a spectral range of 800 nm to 870 nm, with the radiation results between individual sessions of irradiation being monitored. Irradiation takes place using a handheld battery-operated laser light source, which between individual irradiation or after said irradiations can be brought into contact with a base unit that contains a charging device for recharging the batteries in the charging device. The charging device comprises a display which shows the current charge state of the batteries. U.S. Pat. No. 5,464,436 describes various application protocols of the LLLT method with respective irradiation parameters (for example the spacings of irradiation and the duration of irradiation). However, the technique known from U.S. Pat. No. 5,464,436 has a disadvantage in that the setting of irradiation parameters takes place by the user operating the light source, and consequently said setting is highly prone to operator error.

U.S. Pat. No. 6,312,451 describes an LLLT device in which a manually operable handheld laser light source is connected via a supply line to a base unit which comprises a setting device for setting a desired radiation output and a display of the set radiation output. Furthermore, in this technique, the base unit can comprise an input device by means of which a user inputs identification data. Setting the irradiation output can be released or blocked by a control device, which is internal to the base unit, depending on the identification data. In the device known from U.S. Pat. No. 6,312,451, irradiation parameters are specified by the control device of the base unit, but there is a disadvantage in that there is very limited flexibility in the setting of irradiation parameters for various therapeutic tasks. A user is limited to selecting the irradiation output, while application in laser-based acupuncture is possible only to a limited extent.

In the technique described in U.S. Pat. No. 6,312,451, too, an LLLT device comprises a handheld laser light source which is connected to a base unit by way of a supply line. The base unit comprises a control device with a microprocessor and a memory, with which device and memory various irradiation parameters can be specified, such as for example the irradiation output, the selection between pulsed-mode operation and continuous operation, and the duration of treatment. The application options of this LLLT device are limited because the base unit is arranged so as to be stationary. The user must input the desired irradiation parameters into the memory before an application.

A device for laser-based acupuncture is described e.g., in DE 101 07 312 A1. In this device, a laser light source and a circuit for specifying irradiation parameters are accommodated in the housing of a handheld device. This laser treatment device is associated with a disadvantage in that by way of irradiation parameters only the duration of irradiation can be specified, while other variables such as e.g., the pulse frequency during irradiation cannot be specified. Furthermore, the housing of the laser treatment device is large and heavy so that limits are imposed as far as its handling ability is concerned. Lasers for acupuncture treatment are also known from DE 299 07 159 U1 and DE 196 07 174 A1. However, these documents do not provide any references to the control of irradiation parameters.

It has been known to use modulation methods in laser-based acupuncture, in which methods the acupuncture laser is modulated according to a specified protocol. Modulation includes for example pulse generation. As a rule, modulation methods are designated with the names of the persons who first proposed the respective modulation methods, and published by users, such as e.g., the European Academy for Traditional Chinese Medicine “Europaische Akademie für traditionelle chinesische Medizin e.V.”

DE 101 17 297 A1 describes a diagnostic device for pulse wave analysis with a measuring device which comprises a control computer, and with a pulse sensor. Via a connection line, an acupuncture laser can be connected to the measuring device. The control computer in the measuring device can be used to specify irradiation parameters of the acupuncture laser. The application of the diagnostic unit known from DE 101 17 297 A1 is in particular limited to investigating the interaction between acupuncture irradiation and the pulse of the organism exposed to irradiation. As a result of the complex and extensive design of the measuring device, flexible application of the diagnostic device is possible only to a limited extent for acupuncture treatment.

The above-mentioned disadvantages of conventional irradiation devices occur not only in the LLLT and acupuncture devices stated as an example, but also in other handheld laser sources, for example for application in laser surgery or in materials processing.

### OBJECT OF THE INVENTION

It is a first object of the invention to provide an improved irradiation device, with which the disadvantages of conventional irradiation devices can be overcome, and which is characterized in particular by its simplified operation, increased safety and reproducibility as well as an enlarged scope of application. It is a further object of the invention to provide an improved method for exposing the surface of an object to irradiation, in particular for the purpose of irradiation of tissue or materials, with which method the disadvantages of conventional methods are overcome, and which improved method is characterized in particular by its simplified process management and enhanced flexibility in application.

### SUMMARY OF THE INVENTION

From the point of view of device-related aspects, the above-mentioned object is solved by an irradiation device comprising a base unit and at least one light source (in particular a laser light source), whose operating state can be set depending on control signals from a control device which is arranged on the base unit, wherein the control device can be separated (in particular removed) from the base unit. The combination according to the invention, of light source, base unit and control device which can be connected to the base unit is advantageous in that the functionality of the light source and the flexibility of application of the irradiation device can be expanded in a host of different irradiation tasks, in particular in therapeutic treatment such as for example in the case of acupuncture methods. The irradiation device can be prepared without any problems by inserting a control device which has been suitably programmed for the desired application. A user, for example a medical practitioner, can remove the control device and reuse it in another irradiation device. The control device can be used so as to be linked to a particular person, which results in improved safety in the application of the irradiation device.

If according to a first variant of the invention the laser light source is electrically connected to the base unit by way of a line connection, advantageously a voltage source in the base unit can be used for the continuous electrical supply of the laser light source. Advantageously, the line connection can additionally be used for transmission of the control signals to the laser light source.

Particularly advantageously, a further variant provides for the laser light source to be connected wirelessly to the base unit and/or to the control device, and to comprise at least one voltage source for the electrical supply of the laser light source. In this case, advantageously, the flexibility of handling the laser light source is enhanced without its functionality being limited.

If according to a preferred embodiment of the invention the voltage source is rechargeable and for example comprises at least one accumulator, there can be advantages in respect of the operating costs of the irradiation device. Particularly advantageous is the combination according to the invention with the base unit, if said base unit comprises a charging device with which the voltage source, of which there is at least one, is rechargeable. In this ase there is always a suitable charging device available hen the device is used as intended.

If according to a further modification of the invention the body of the base unit comprises at least one accumulator holder which is connected to the charging device, advantageously, individual accumulators as voltage sources for the laser light source can be recharged even if said laser light source is not connected to the base unit.

A preferred embodiment to the invention provides for the base unit moreover to comprise a coupling device for connection to, and mechanical holding of, the laser light source (first coupling device). In this case, advantages can result for safe holding of the laser light source off-time the radiation operation and/or for simplified connection of the laser light source to the charging device.

If a further embodiment of the invention provides for the base unit to comprise a (second) coupling device for disconnectable connection to, and mechanical holding of, the control device, this can have advantages in relation to fast and safe attachment of the control device to the base unit. If the second coupling device is not only equipped for mechanical connection but at the same time also provides an electrical interface, the use of the irradiation device according to the invention is simplified.

In particular in the case of a wireless connection between the laser light source and the base unit and/or the control device, the laser light source preferably comprises a laser send-receive device (first send-receive device), while a base send-receive device (second send-receive device) is integrated in the base unit or the control device. If the wireless connection is a Bluetooth or infrared connection, this can result in advantages from the application of data transmission techniques which are available per se.

Advantageously, the control signals that are transmitted from the control device to the light source in a line-bound or wireless manner can directly be used for activating the light source. Preferably, pulsed operation is provided in which the control signals represent a particular pulse sequence in accordance with a desired treatment protocol, wherein the output of the radiated light at the light source can be selected by selecting between various output stages. According to the invention, the wireless connection or line connection between the light source or the control device is thus used for transmitting information which as a pulse sequence represents the desired treatment protocol.

A further preferred embodiment of the invention is characterized in that the control device, which is separable from the base unit, comprises a programmable microprocessor. The control device can in particular comprise a portable computer which can be operated independently of the base unit and of the laser light source; such computers being available for example in the form of so-called pocket PCs. Such a design considerably expands the functionality and ease of operation of the irradiation device.

If the irradiation device according to the invention comprises a display, this can provide advantages to the user in relation to the monitoring of operating states and the provision of further information. Preferably, the display forms part of the control device and together with said control device can be separated from the base unit. In this case, advantageously, information concerning the programming state of the control device can also be displayed in the separated state. As an alternative or in addition, the display is provided on the body of the base unit. Advantageously, in this case further operating states of the base unit, for example the ready state of the charging device, can be displayed.

Particular advantages for convenient operation result if the display comprises a touch screen. This advantage is in particular significant for therapeutic application of the irradiation device because during preparation or during the treatment, which often is carried out in confined spaces without sufficient space for a keyboard, a great body of information arises, for example about the physiological state of a patient, which body of information can in this way be entered into the control device in a particularly effective way.

The laser light source of the irradiation device according to the invention can comprise at least one manually operable switch, for example an activation key or a touch pad, which can advantageously be used as a main switch or emergency switch and/or as an operational mode selector switch so that expanded functionality of the irradiation device can result. If according to a further modification the laser light source comprises a pilot beam laser, advantages for visualising the path of the laser light emanating from the laser device can result. For adaptation to specific radiation tasks, e.g., to specific object surfaces, the laser light source can comprise a beam guidance device for adapting the emitted laser light to the geometric conditions at the place of irradiation. In this case, advantages for increased effectiveness of treatment may result.

Advantageously, operational safety of the irradiation device according to the invention can be enhanced if an acoustic signal transmitter, such as for example a buzzer or a loudspeaker, is provided, for example to signal unintended or dangerous operational states of the laser light source.

As far as the method is concerned, the above-mentioned object is solved in that for exposing the surface of an object to radiation, in particular with the irradiation device according to the invention, first the control device of the base unit is provided, then is connected to said base unit and is activated, after which the laser light source is activated for exposing the object surface to radiation. In contrast to conventional radiation methods, the invention provides a particular advantage in that, by way of suitable programming of the control device, the irradiation conditions and if applicable further information concerning the person to be treated can already be prepared by the user before irradiation treatment is carried out. For treatment, the user, e.g., the medical practitioner, can then insert the prepared control device into the base unit and activate it.

According to a preferred embodiment of the method according to the invention, activation includes the selection of at least one of the following parameters: radiation output, duration of irradiation, frequency, and clock-pulse ratio of pulsed radiation, or the selection of a tutorial program. Thus, in contrast to conventional treatment methods, advantageously a larger number of variable parameters are provided, under which parameters the desired irradiation conditions can be carried out.

Advantageously, according to preferred variants of the invention, frequency selection can comprise the selection of a pre-programmed frequency, a mixed frequency of pre-programmed frequencies and/or a free frequency. According to the invention, in this way the modulation method for LLLT treatment or laser-based acupuncture, which methods are known per se, can thus be carried out easily. According to a preferred variant of the method according to the invention, the step of activating the control device comprises signal transmission to the laser light source. Advantageously, the laser light source is controlled at increased operational safety completely according the specified operational parameters. Manual operating errors are thus precluded. A further advantage consists of the laser light source being able to be made operational almost concurrently with activation of the control device.

The irradiation of biological tissue, in particular the irradiation of the skin surface of a person to be treated, such as for example of a patient, is a preferred application of the invention. Advantageously, the procedures, which are known per se, of the LLLT methods or of the acupuncture methods can be implemented. For example, scar follow-up treatment, acupuncture stimulation or wound healing can be provided. Further applications relate to laser-based or optical surgery. The irradiation device according to the invention can be used as a laser scalpel (cutting laser). Medical applications also include the field of dermatology, e.g., the removal of lentigines, also known as age spots, or the removal of tattoos; the field of dental medicine, e.g., wound healing or root treatment; or the field of ophthalmology, e.g., the correction of defective vision.

There are also applications in materials processing using thermal effects, such as for example the treatment of surface layers or the curing of polymer materials.

The irradiation device according to the invention can in particular be used as a laser therapy unit for exposing small or medium sized skin areas or body parts to radiation (so-called biostimulation). Advantageously, the laser is used for frequency diagnostics (so-called RAC/“reflex auriculo cardial”) and frequency therapy in acupuncture or other medical fields, or for transmitting frequency information and photon energy. Apart from frequency diagnosis, the irradiation device can also be used for electronic diagnosis (for example measuring and analysing acupuncture points or changes in skin resistance).

Furthermore, important characteristics of the irradiation device according to the invention include the provision of the laser light source as a compact, programmable mobile handheld device, independent of electricity means, with said device interacting in a user-friendly way with the base unit as a station for charging the accumulators, the storage of the laser and of a computer (type: e.g., iPAQ 1940, manufactured by Hewlett Packard, with an integrated Bluetooth interface).

### BRIEF DESCRIPTION OF THE DRAWINGS

Further advantages and details of the invention are provided in the following description of the enclosed drawings. The following are shown:

FIG. 1: a diagrammatic representation of an irradiation device according to the invention;

FIG. 2: a perspective view of an embodiment of a base unit used according to the invention;

FIG. 3: a diagrammatic top view of an embodiment of a laser light source used according to the invention;

FIG. 4: illustrations of an embodiment of a control device used according to the invention and of the information displayed by means of said control device;

FIG. 5: a further embodiment of a laser light source used according to the invention; and

FIGS. 6 & 7: diagrammatic illustrations of beam guidance devices used according to the invention.

### DETAILED DESCRIPTION OF PREFERRED EMBODIMENTS OF THE INVENTION

Below, the implementation of the invention is explained using the general representation according to FIG. 1, with exemplary reference to the combination of a laser light source in the form of a manually operatable, handheld light pen (or: laser pen) and a pocket computer which can be connected to a base unit. It should be emphasised that the implementation of the invention is not limited to the details of this combination, but instead that numerous variants, in particular relating to the design of the light source and the control device as well as relating to the design and functionality of the base unit, can be provided. For example, it is not mandatory for the light source to be equipped to emit laser light. For particular treatment purposes it can be adequate if non-coherent light from a classical thermal light source (incandescent light) or a light emitting diode is used. Furthermore, in particular instead of the pocket computer, a control device of some other design, comprising an integrated microprocessor and an input device for programming the microprocessor can be provided, which can be connected to the base unit by way of an interface. Furthermore, in a way which is different from that shown in the embodiment shown below, several laser light sources can be combined with the base unit, with said laser light sources being used in sequence by one user, or alternatively by several users concurrently. Depending on the desired application of the irradiation device, it can be equipped for emitting one or several wavelengths in the entire spectral range from ultraviolet to visible light right through to infrared light.

According to FIG. 1, an irradiation device 100 comprises a laser light source 10 and a base unit 20. The laser light source 10 comprises a housing 11 in or on which the laser device 12 for emitting laser light, the voltage source 13 for supplying electricity to the laser light source 10, at least one activation key 14, and the first send-receive device 15 are arranged. The base unit 20 comprises a device body 21; the control device 22, which on the second coupling device 23 is detachably connected to the device body 21; the first coupling device 24 for temporary holding of the laser light source 10, the second send-receive device 25 and a accumulator holder 26. Reference number 27 relates to optionally provided display devices and/or input devices, e.g., for a charging device (not shown) which is integrated in the base unit 20.

The housing 11 of the laser light source 10 is of elongated shape, for example cylindrical shape, comprising a suitably designed surface, which in particular can comprise grip elements for safe handling of the laser light source 10 and/or information lettering. The laser device 12 is provided at the front of the housing 11. It comprises a laser diode (output: 50 mW to 5 W or more) which is designed for emission in the spectral range of 620 nm through to the infrared spectral range (e.g., 900 nm). For applications in laser surgery, an output of 10 W or more can be provided. The voltage source 13 comprises two accumulator cells which can be inserted into the laser light source 10 from the rear of the housing 11. To this effect, an opening with a cover and a snap-in device for holding the accumulators in place is provided at the rear.

The first send-receive device 15 comprises an aerial designed for implementing Bluetooth connections, and an associated processing circuit for signal processing the control signals received from the control device 22.

The operating state of the laser device 12 can be set depending on the control signals of the control device 22. This means that the control signals are directly provided as switching signals for the laser device 12. For example, a pulse train with modulation which corresponds to the desired modulation of the laser diode is transmitted. As an alternative, the control signals can code certain operating states of the laser device 12, which operating states are stored in a memory which optionally is additionally provided in the light source.

The device body 21 of the base unit 20 also forms a housing, in which the charging device and a mains voltage supply unit are accommodated, and on whose outside the first and second coupling devices 23, 24 and the accumulator holder 26 are arranged.

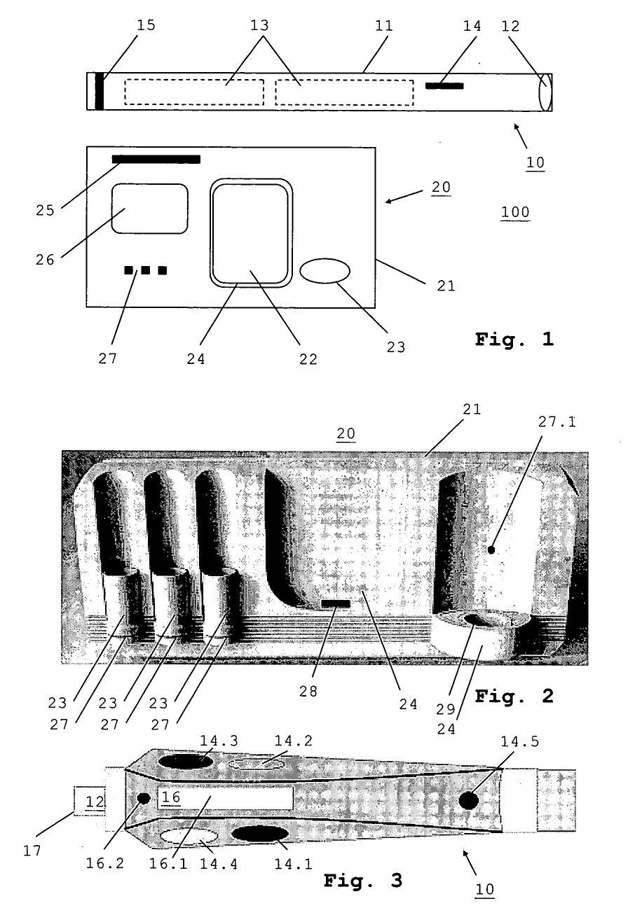

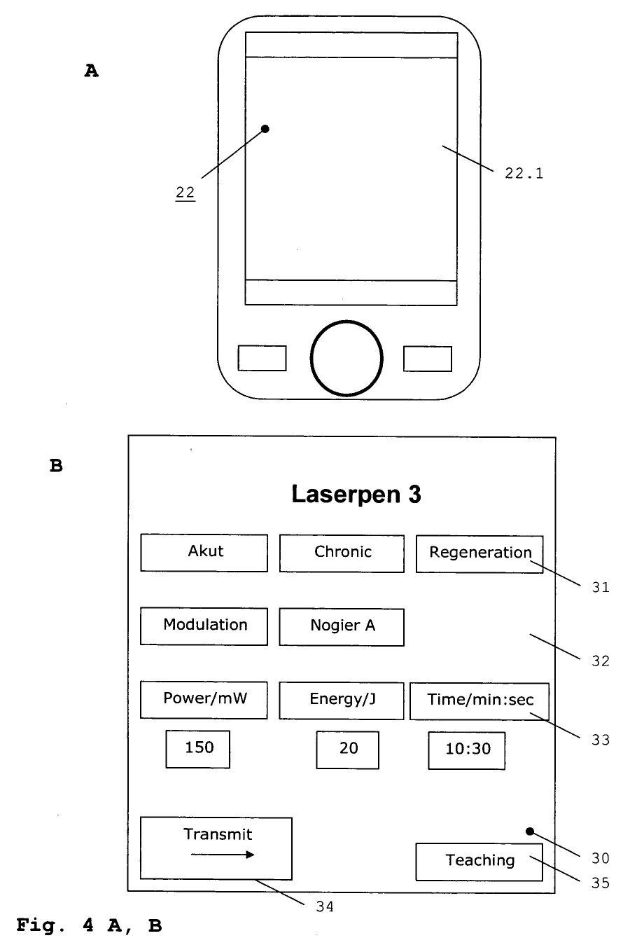

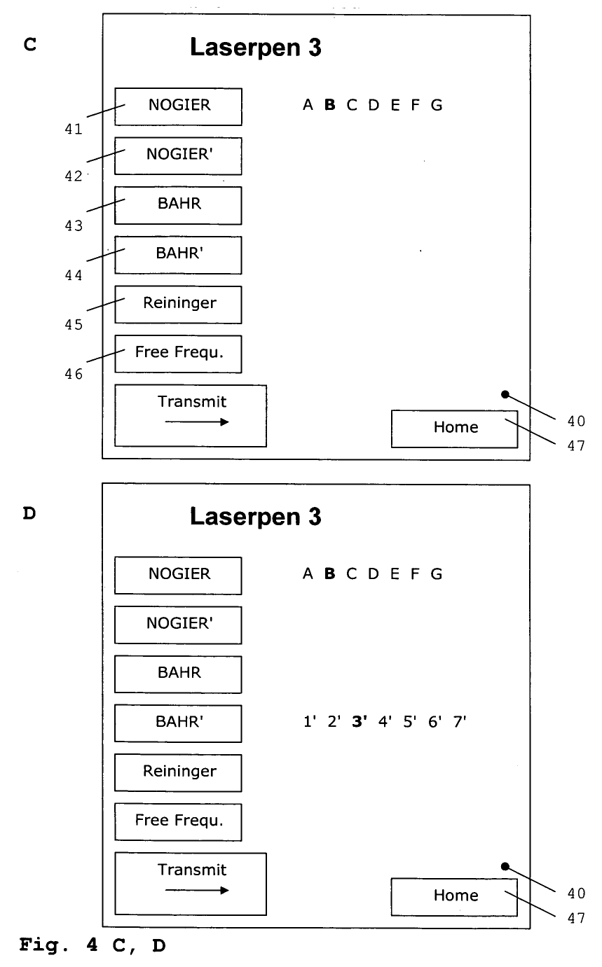

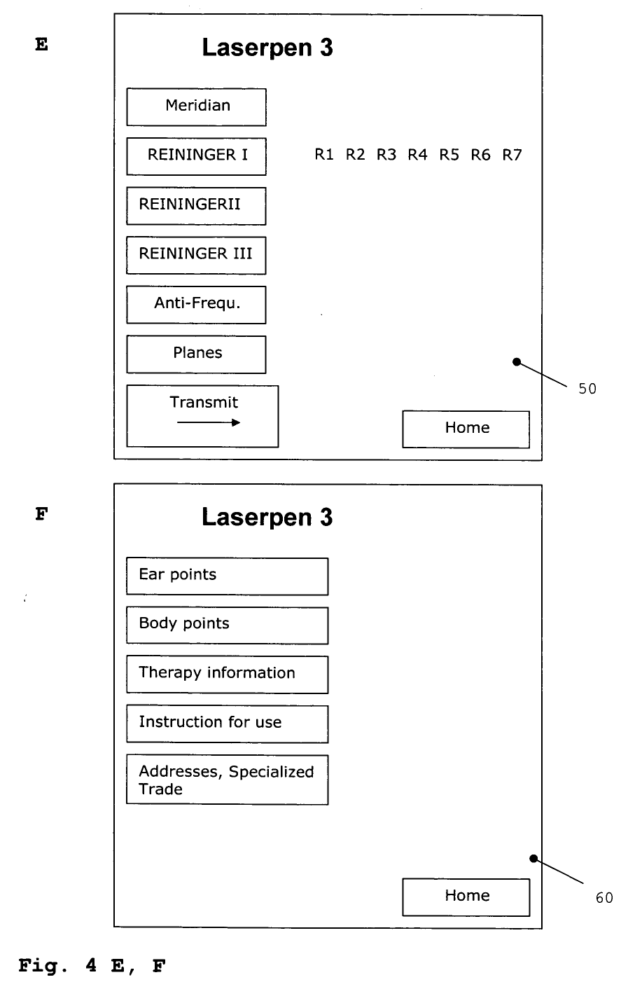

If, as is shown in the embodiment according to the invention as shown in FIG. 1, the second send-receive device 25 is integrated in the base unit 20, the second coupling device 24 comprises an interface for electrically connecting the control device 22 to a signal processing circuit (not shown) of the device 25. As an alternative, the second send-receive device 25 can form part of the control device 22, as will be explained below with reference to FIGS. 2 and 4. In this case, advantageously there is no need to create an additional interface for signal transmission between the control device 22 and the base unit 20. In both embodiments, at least one electrical connection for recharging an internal voltage source of the control device 22 can be provided on the second coupling device.

Further details of the outside of the base unit 20 are illustrated by way of example in FIG. 2. The shape of the first coupling device 23 matches the shape of the laser light source 10 that is used. As a first coupling device 23, the device body 21 comprises a circular projecting part comprising a central receptacle 29 into which the laser light source 10 can be plugged at its front or rear. In the region of the projecting part 23, the device body 21 comprises a cylindrical recess so that taking of the plugged in laser source is facilitated. The second coupling device 24 forms a receptacle with an inner shape which matches the outer shape of the control device, such as e.g., a pocket computer 22 according to FIG. 4a. The second coupling device 24 comprises a fastening device, such as e.g., a snap-in device 28 for holding in place the inserted control device.

In the embodiment illustrated in FIG. 2, three accumulator holders 26 are provided, each of which comprises a charging display 27. At the top of the accumulator holders 26, the device body 21 comprises cylindrical recesses, each of which forms a charging well for accumulators.

Reference number 27.1 refers to a sensor device for monitoring the operational ability of the laser device 12 of the laser light source 10. Since in numerous therapeutic applications the laser device 12 is operated in the infrared spectral range, i.e., in the non-visible spectral range, the sensor device 27.1 serves for a function test. For example an infrared detector can be provided as a sensor device 27.1. If the laser radiation is directed towards the sensor device 27.1, with proper laser function an acoustic signal can sound, for example in rhythm with the pulse frequency of the laser. As an alternative, the sensor device 27.1 can be a thermal sensor made of a material which reacts to infrared radiation by way of a colour change.

FIG. 3 shows further details of the laser light source 10 in which instead of a single activation key several keys or switches 14.1 to 14.5 are provided which have the following functions: key 14.1 is a switch for switching the laser light source 10 on or off. The switching device (e.g., keys) 14.2 and 14.3 is used for preselecting various output ranges (or output stages) for operating the laser device 12. For example, activating key 14.2 results in operation starting at 5 mW, while activating key 14.3 results in operation starting at 50 mW. A further output stage, e.g., of 5 W, can be provided. Key 14.4 is used to switch between various modulation frequencies when operating the laser device 12. Finally, switch 14.5 is a key switch which can only be activated by an authorised user with the appropriate key.

In the embodiment of the laser light source 10 shown in FIG. 3, furthermore a display device 16 is provided which comprises an information display 16.1 and a status display 16.2. The information display 16.1 is for example an alphanumeric display which e.g., shows operation parameters of the irradiation device. The information display 16.1 for example shows the output in mW, the energy (Joule) (indication of the energy already transmitted), a therapy time (Joule) (with remaining-time display), and/or the current modulation frequency.

The status display 16.2 serves as a warning display when the laser device 12 is active. When operating the laser light source 10 it can be provided for the status display 16.2, e.g., in the form of a green light emitting diode, to first blink during a specified lead time (e.g., 2 seconds) after the start of operation of the laser device 12 as a warning of the impending laser radiation, and for said green light emitting diode to then light continuously for the duration of operation of the laser device 12. Accordingly, prior to the start of operation of the laser device, the pilot beam can mark the point of impact of the laser beam.

The laser device 12 comprises an irradiation laser, e.g., the above-mentioned laser diode, and a pilot laser which emits in the visible spectral range and is aligned identically with the direction of radiation of the irradiation laser. For acupuncture applications the laser diode is typically operated at a distance of at least 30 mm. In surgical applications, smaller distances for example of 15 mm or below are usual. Typical output densities in thermal or ablative applications of the irradiation device according to the invention range from mW/cm² (biostimulation), to kW/cm² (coagulation) right through to MW/cm² (evaporation) or even GW/cm² (photoablation).

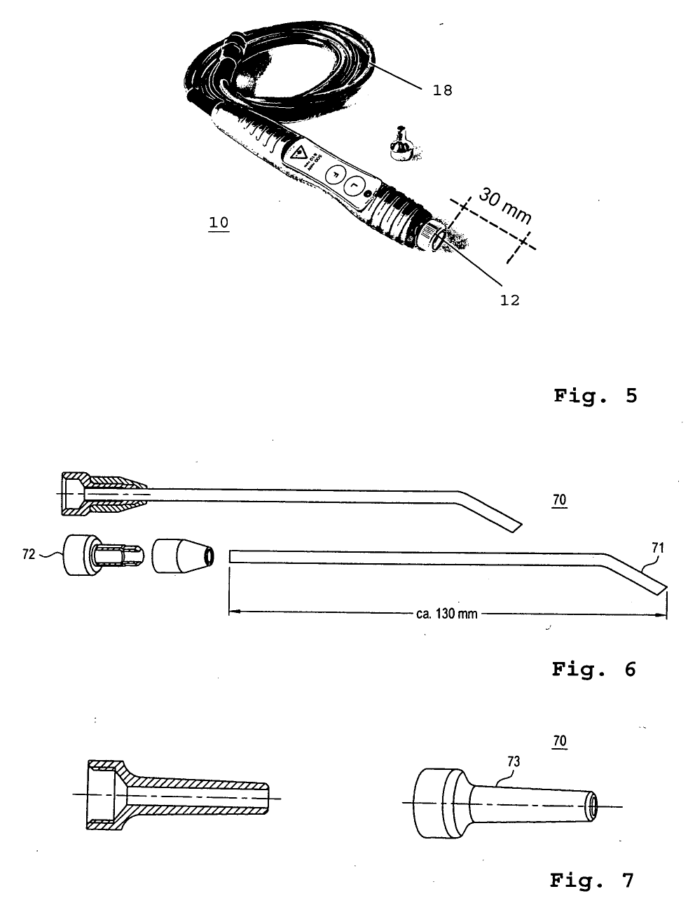

The laser device 12 provides for a connection piece 17 which is used to hold beam guidance devices (see below, FIGS. 6 and 7).

By way of example, FIG. 4 shows a design of the control device 22 as a pocket computer comprising a touch screen 22.1 (FIG. 4A), wherein the FIGS. 4B to 4F by way of example illustrate the contents and activation elements displayed on the control device 22.

After the control device 22 has been switched on, the start image 30 according to FIG. 4B appears, providing three selection lines 31, 32 and 33; a transmission field 34; and
a help key 35 (“Teaching”). In the first selection line 31, a selection can be made between various preprogrammed modulation frequencies, e.g., for the applications “Acute”, “Chronic” and “Regeneration”. In the second selection line 32 a particular modulation method is selected. In the example shown, the modulation method “Nogier A” has been selected. In the third selection line 33, the output, the energy, and the duration of radiation can each be preselected in the individual fields.

By touching a keypad, the respective function is activated. For example, the key pad changes colour or a new window appears where further details can be set. After completion of data input, and the key 34 has been activated, the data are sent to the laser light source 10 as control signals.

FIG. 4C illustrates the selection of a preset modulation frequency in a selection window 40, which in several selection lines 41, 42 etc. lists various default frequencies, wherein if applicable a particular frequency can be selected from a frequency range. In the example shown, for example frequency B of the modulation frequency “Nogier” is activated.

When the selection line 46 is activated, a new keypad opens for setting freely selectable frequencies. The keypad 47 is used for returning to the start image 30 according to FIG. 4B.

FIG. 4D illustrates the setting of mixed frequencies. To this effect various modulations and associated frequencies are activated on the selection image 40. In the example shown, the frequencies “Nogier B” and “Bahr 3” are intermixed.

If in the selection image 40 according to FIG. 4C the fifth selection line 45 is selected, then the lower level display 50 for the modulation “Reininger” opens with the respective selection options.

Finally, FIG. 4F illustrates the teaching display 60 which appears when the keypad 35 according to FIG. 4B appears. In the various selection lines, tutorial programs and user instructions as well as more detailed information can be called up.

The following tables illustrate various frequencies of modulation according to Nogier, Bahr and Reininger, which frequencies with corresponding activation of the control device 22 can be transmitted as control signals for operating the laser light source 10.

Frequencies according to Nogier/Bahr:

| Frequencies | | | Frequencies | | | | | |
|---|---|---|---|---|---|---|---|---|
| according to | | | according to | | | Frequencies | | |
| Nogier: | | | Nogier: | | | according to Bahr: | | |
| Non-exponentiated | | | Exponentiated | | | | | |
| A = | 2.28 | Hz | A′ = | 292 | HZ | B1 = | 599.50 | Hz |
| B = | 4.56 | Hz | B′ = | 584 | Hz | B2 = | 1199.00 | Hz |
| C = | 9.12 | Hz | C′ = | 1168 | Hz | B3 = | 2398.00 | Hz |
| D = | 18.25 | Hz | D′ = | 2336 | Hz | B4 = | 4796.00 | Hz |
| E = | 36.48 | Hz | E′ = | 4672 | Hz | B5 = | 9592.00 | Hz |
| F = | 73.00 | Hz | F′ = | 9344 | Hz | B6 = | 19184.00 | Hz |
| G = | 146.00 | Hz | G′ = | 18688 | Hz | B7 = | 38368.00 | Hz |

| | | | |
|---|---|---|---|
| U = | 1.14 | Hz | Continuous beam |

10 positions
freely
programmable
Frequency 01
to
frequency 10

Reininger frequencies: (Reininger I, II, III,
Meridian frequencies, “antifrequencies”, frequencies of the
levels, frequencies viral/bacterial)

| | | |
|---|---|---|
| Reininger I | Reininger II | Reininger III |

| | | | | | | | | |
|---|---|---|---|---|---|---|---|---|
| RI 1 | 132 | Hz | RII 1 | 113 | Hz | RIII 1 | 114 | Hz |
| RI 2 | 264 | Hz | RII 2 | 226 | Hz | RIII 2 | 228 | Hz |
| RI 3 | 528 | Hz | RII 3 | 452 | Hz | RIII 3 | 456 | Hz |
| RI 4 | 1056 | Hz | RII 4 | 904 | Hz | RIII 4 | 912 | Hz |
| RI 5 | 2112 | Hz | RII 5 | 1808 | Hz | RIII 5 | 1824 | Hz |
| RI 6 | 4224 | Hz | RII 6 | 3616 | Hz | RIII 6 | 3648 | Hz |
| RI 7 | 8448 | Hz | RII 7 | 7232 | Hz | RIII 7 | 7296 | Hz |

| Meridian | | | Anti-frequencies | Frequencies of the |
|---|---|---|---|---|
| frequencies | | | Psyche 129 Hz | levels |
| Le = | 442 | Hz | Anti-psyche | Melatonin-HWF |
| Ma = | 471 | Hz | 4221 Hz | 1025 Hz |
| He = | 497 | Hz | Vegetativum 112 Hz | VIP1/2 1808 Hz |
| KS = | 530 | Hz | Anti-vegetativum | TUP 7232 Hz |
| Di = | 553 | Hz | 3665 Hz | LG/KG 456 Hz |
| Gb = | 583 | Hz | Addiction 112 Hz | MdO 7296 Hz |
| Ni = | 611 | Hz | Anti-addiction | M-HWP 4353 Hz |
| Bl = | 667 | Hz | 3305 Hz | M-HWP 3200 Hz |
| MP = | 702 | Hz | Carcinoma 108 Hz | Valium-HWF |
| 3E = | 732 | Hz | Anti-carcinoma | 1153 Hz |
| Du = | 791 | Hz | 3534 Hz | Psych. blockade |
| Lu = | 824 | Hz | Pain 119 Hz | point 228 Hz |

| | |
|---|---|
| Anti-pain 3894 Hz | Base-S 528 Hz |
| Inflammation | Base-T 132 Hz |
| 128 Hz | V-HWP 2817 Hz |
| Anti-inflammation | Konakion-HWF |
| 4189 Hz | 3968 Hz |
| Allergy 3648 Hz | K-HWP 4864 Hz |
| Anti-allergy | K-HWP 2432 Hz |
| 933 Hz | |
| Tinnitus 125 Hz | |
| Anti-tinnitus | |
| 4090 Hz | |
| General 384 Hz | |

| | |
|---|---|
| Frequencies | HWF = Reference |
| Viral/bacterial | point |
| Viral 1408 | |
| Anti-viral 360 Hz | |
| Bacterial 1664 Hz | |
| Anti-bacterial 425 Hz | |

Modulation frequencies ranging from 0.2 to 999.99 Hz can be set.

FIG. 5 illustrates a modified embodiment of the laser light source 10 according to the invention in which an electrical connection line 18 for connection to the base unit is provided. In this variant, operation of the control device as well as signal transmission to the laser light source 10 can take place analogously to the procedures described above.

FIGS. 6 and 7 illustrate several variants of beam guidance devices 70 provided according to the invention, which beam guidance devices 70 can be placed onto the holding device 17 of the laser light source 10 (see FIG. 3). The beam guidance device 70 according to FIG. 6 is used as a dental applicator which comprises a fibre-optic light guide 71 (approximately 13 cm in length) made of glass in a stainless steel shell, as well as add-on pieces 72. FIG. 7 illustrates a point-applicator in diagrammatic sectional view and perspective view. The diameter of the exit bore hole of the point applicator 73 is e.g., 4 mm.

The characteristics of the invention disclosed in the above description, in the claims and drawings can be of importance either individually or in combination for implementing the invention in its various embodiments.

1. An irradiation device comprising:
at least one laser light source with a housing in which a laser device is arranged;
a base unit comprising a device body; and
a control device,
wherein an operating state of the laser device can be set depending on control signals of the control device, and the control device is arranged so as to be separable from the device body.

2. The irradiation device according to claim 1, wherein the at least one laser light source is connected to the base unit by way of a line connection, and the base unit comprises a voltage source for supplying electricity to the laser light source.

3. The irradiation device according to claim 1, wherein the at least one laser light source is connected to the base unit by way of a wireless connection, and comprises at least one voltage source for supplying electricity to the laser light source.

4. The irradiation device according to claim 3, wherein the voltage source comprises a rechargeable voltage source.

5. The irradiation device according to claim 4, wherein the base unit comprises a charging device with which the voltage source, of which there is at least one, can be recharged.

6. The irradiation device according to claim 5, wherein the device body comprises at least one accumulator holder connected to the charging device.

7. The irradiation device according to claim 1, wherein the base unit comprises a first coupling device for connecting the laser light source.

8. The irradiation device according to claim 1, wherein the base unit comprises a second coupling device for connecting the control device.

9. The irradiation device according to claim 3, wherein the laser light source comprises a first send-receive device, and the base unit comprises a second send-receive device adapted to establish a wireless connection with the first send-receive device.

10. The irradiation device according to claim 9, wherein the wireless connection comprises a Bluetooth connection or an infrared connection.

11. The irradiation device according to claim 1, wherein the control device comprises a programmable micro-processor.

12. The irradiation device according to claim 1, further comprising a display.

13. The irradiation device according to claim 12, wherein the display forms a part of the control device.

14. The irradiation device according to claim 12, wherein the display forms a part of the device body.

15. The irradiation device according to claim 12, wherein the display comprises a touch-sensitive display screen.

16. The irradiation device according to claim 1, wherein the at least one laser light source comprises an activation key.

17. The irradiation device according to claim 1, wherein the at least one laser light source comprises a pilot beam laser.

18. The irradiation device according to claim 1, wherein the at least one laser light source comprises at least one of a beam guidance device adapted to align laser light emitted from the laser device, and an acoustic signal transmitter.

19. The irradiation device according to claim 1, wherein the at least one laser light source comprises a switching device adapted to select various outputs during operation of the laser device.

20. A method for exposing an object to radiation, with an irradiation device according to claim 1, comprising the steps of:
providing the control device on the body of the base unit;
activating the control device; and
activating the at least one laser light source so that a surface of the object is exposed to radiation.

21. The method according to claim 21, wherein the step of activating the control device comprises at least one of the following steps:
selecting a modulation frequency;
selecting an output;
selecting a duration of irradiation; and
selecting a tutorial program.

22. The method according to claim 21, wherein selection of a preprogrammed modulation frequency comprises at least one of the following steps:
selecting a modulation frequency;
selecting a combination frequency; and
selecting a free frequency.

23. The method according to claim 20, wherein activation of the control device involves data transmission to the at least one laser light source.

24. The method according to claim 20, wherein the object subjected to radiation comprises biological tissue or a material.

25. The method of claim 20, wherein the object is exposed to radiation in conjunction with at least one of the following methods:
LLLT method;
acupuncture;
laser surgery;
skin treatment in dermatology;
root treatment in dentistry;
scar follow-up treatment; and
materials treatment.
